(19) United States
(12) Patent Application Publication
(10) Pub. No.: US 2013/0129656 A1
(43) Pub. Date: May 23, 2013

(54) "GREEN" DIESTER AMINES AND PERSONAL CARE PRODUCTS

(71) Applicants: Abel G. Pereira, Bridgewater, NJ (US); Erik Gunderman, Edison, NJ (US); Duane St. Amour, Parlin, NJ (US); Farahdia Edouard, Edison, NJ (US)

(72) Inventors: Abel G. Pereira, Bridgewater, NJ (US); Erik Gunderman, Edison, NJ (US); Duane St. Amour, Parlin, NJ (US); Farahdia Edouard, Edison, NJ (US)

(73) Assignee: CRODA, INC., Edison, NJ (US)

(21) Appl. No.: 13/814,631

(22) PCT Filed: Aug. 8, 2011
(86) PCT No.: PCT/US11/46912

§ 371 Date: Feb. 6, 2013

Related U.S. Application Data
(60) Provisional application No. 61/372,297, filed on Aug. 10, 2010.

(51) Int. Cl. C07C 219/06

(52) U.S. Cl. CPC C07C 219/06
USPC 424/70.1; 514/547; 554/110

(57) ABSTRACT

Diester tertiary amines and quats and their methods of synthesis are disclosed. Also disclosed are personal care compositions comprising the disclosed compounds.

### CROSS-REFERENCE TO RELATED APPLICATIONS

This application claims the benefit of the filing date of U.S. Provisional Patent Application No. 61/372,297, filed Aug. 10, 2010, the disclosure of which is hereby incorporated herein by reference.

### BACKGROUND OF THE INVENTION

There are many known types of compounds bearing tertiary or quaternary amine groups. These compounds can be tremendously useful in many different fields, including in personal care products. As our understanding of technology, chemistry, and the environment expand, there is an increased sensitivity to the interrelationships between them and our surroundings. Accordingly, there is room to improve the chemistry of known classes of chemicals to make them more environmentally friendly.

WO 2004/093834 discloses personal care products containing a mixture of at least one personal care ingredient and one diester quaternary ammonium compound.

### BRIEF SUMMARY OF THE INVENTION

In one embodiment of the present invention, Applicants have discovered a compound of Formula (I):

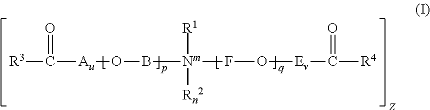

wherein R¹ and R² are independently selected from branched or straight chain, substituted or unsubstituted, saturated or unsaturated alkyl, cyclic or aromatic groups including between about 1 and about 22 carbon atoms;

B and F are independently a straight chain or branched alkyl group having between 2 and 8 carbon atoms,

p and q are independently 0 or 1;

A and E are independently selected from a straight chain or branched alkoxy group having between 1 and 8 carbon atoms;

u and v may be the same or different and are each an integer greater than 1;

R³ and R⁴ are independently selected from branched or straight chain, substituted or unsubstituted, saturated or unsaturated alkyl, cyclic or aromatic groups including between about 1 and about 36 carbon atoms;

Z is a counterion or salt former and included only in cases where the compounds of Formula (I) are a salt or quat as appropriate (hereinafter “counterion”);

n is 0 or 1;

m is a lone pair of electrons or a positive charge;

with the proviso that if n is 0, then m is a lone pair of electrons and there is no counterion (Z); and if n is 1, then m is a positive charge and the counterion (Z) is present; and with the proviso that if Aᵤ and Eᵥ each independently represent blocks containing propoxy and ethoxy groups, the number of propoxy groups is greater than the number of ethoxy groups.

In some embodiments, the compounds of Formula (I) have a biodegradability of at least about 50% as measured by a OECD-301B testing methodology. In other embodiments, the compounds of Formula (I) have an aquatic toxicity of at least about 10 mg/L as measured by OECD-202: Daphnia sp. Acute Immobilization Test methodology. In yet other embodiments, the compounds of Formula (I) have a biodegradability of at least about 50% as measured by OECD-301B testing methodology and an aquatic toxicity of at least about 10 mg/L as measured by OECD-202: Daphnia sp. Acute Immobilization Test.

In some embodiments, R² and R² are independently selected from lower alkyl groups having between about 1 and about 8 carbon atoms. In other embodiments, R² and R² are independently selected from methyl or ethyl.

In some embodiments, R³ and R⁴ are independently selected from saturated or unsaturated alkyl groups having between about 18 and about 24 carbon atoms.

In some embodiments, Aᵤ and Eᵥ are comprised of blocks of alkoxy groups. In other embodiments, Aᵤ and Eᵥ are independently comprised of blocks of ethoxy groups and/or propoxy groups (linear or branched). In yet other embodiments, Aᵤ and Eᵥ independently comprise mixed blocks of ethoxy and propxy groups. In yet further embodiments, the ratio of propoxy groups to ethoxy groups when mixed blocks are present (in either Aᵤ or Eᵥ, independently) range from 3:2 to 2:3. In even further embodiments, Aᵤ or Eᵥ are each —[(O—CH₂CH₂)₃]—[(O—CH(CH₃)CH₂)₂].

In some embodiments, A and E are independently selected from ethoxy and propoxy. In other embodiments, A and E are both propxy. In yet other embodiments, A and E are both propoxy and u and v independently range from 2 to 5. In yet further embodiments, R² and R² are independently methyl or ethyl, R³ and R⁴ are independently selected from saturated or unsaturated alkyl groups having between 18 and 24 carbon atoms, Aᵤ and Eᵥ are independently blocks of ethoxy or propoxy groups or combinations thereof.

In another embodiment of the present invention, Applicants have discovered a personal care product comprising at least one personal care ingredient and a compound of Formula (I). The at least one personal care ingredient can include, without limitation, any solvent, surfactant, conditioner, pigment, UV protector or UV protecting group, color, fragrance, dye, excipient or additive useful in formulating personal care products such as, without limitation, cosmetics, sun-screens and sun-blocks, shampoos, skin creams, gels or lotions, conditioners, softeners and the like. In some embodiments, the personal care compositions comprise one or more additives. In other embodiments, the personal care composition comprises an additional quaternary or tertiary amine different from that of Formula (I). The compositions may be in the form of a flake pastillate. These personal care products are “greener” because of the use of compounds of Formula (I) relative to identical products with some other diester amine.

In another embodiment of the present invention, Applicants have discovered a compound of Formula (II):

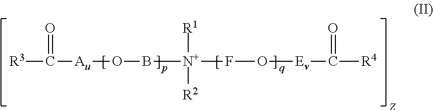

wherein R¹ and R² are independently selected from branched or straight chain, substituted or unsubstituted, saturated or unsaturated alkyl, cyclic or aromatic groups including between about 1 and about 22 carbon atoms;

B and F are independently a straight chain or branched alkyl group having between 2 and 8 carbon atoms,

p and q are independently 0 and 1;

A and E are independently selected from a straight chain or branched alkoxy group having between 1 and 8 carbon atoms;

u and v may be the same or different and are each an integer greater than 1;

R³ and R⁴ are independently selected from branched or straight chain, substituted or unsubstituted, saturated or unsaturated alkyl, cyclic or aromatic groups including between about 1 and about 36 carbon atoms;

Z is a counterion or salt-former; and

with the proviso that if Aᵤ and Eᵥ each independently represent blocks containing propoxy and ethoxy groups, the number of propoxy groups is greater than the number of ethoxy groups.

In some embodiments, Aᵤ and Eᵥ of Formula (II) are comprised of blocks of alkoxy groups. In other embodiments, Aᵤ and Eᵥ are comprised of blocks of ethoxy groups or propoxy groups (linear or branched) or any combinations thereof. In yet other embodiments, Aᵤ and Eᵥ comprise mixed blocks of ethoxy and propxy groups. In some embodiments, the ratio of propoxy groups to ethoxy groups (in either Aᵤ or Eᵥ, independently) range from 3:2 to 2:3.

In some embodiments, A and E of Formula (II) are independently selected from ethoxy and propoxy. In other embodiments, A and E are both propxy. In yet other embodiments, A and E are both propoxy; and u and v independently range from 2 to 5.

In some embodiments, the compounds of Formula (II) have a biodegradability of at least about 50% as measured by OECD-301B testing methodology. In other embodiments, the compounds of Formula (I) have an aquatic toxicity of at least about 10 mg/L as measured by OECD-202: Daphnia sp. Acute Immobilization Test methodology. In yet other embodiments, the compounds of Formula (I) have a biodegradability of at least about 50% as measured by OECD-301B testing methodology and an aquatic toxicity of at least about 10 mg/L as measured by OECD-202: Daphnia sp. Acute Immobilization Test testing methodology.

In some embodiments, the compounds of Formula (II) are combined with a personal care ingredient to form a personal care composition, such as a shampoo or conditioner. The personal care composition may include additives or solvents as described herein.

In some embodiments, Aᵤ and Eᵥ of Formulas (I) or (II) are independently selected from the group having the Formula (III):

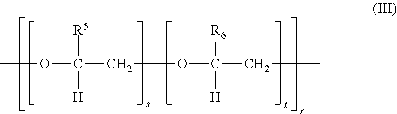

where R⁵ and R⁶ are independently H or CH₃;

s and t are independently 0 or an integer ranging between 1 and 10; and

r is an integer between 1 and 10.

Not shown in Formula (III), but nevertheless contemplated, are embodiments where any propoxy group is linear, i.e. n-propyl.

In another embodiment, Applicants have discovered a compound of Formula IV:

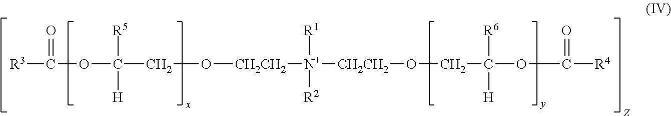

wherein R¹ and R² are independently selected from branched or straight chain, substituted or unsubstituted, saturated or unsaturated alkyl, cyclic or aromatic groups including between about 1 and about 22 carbon atoms;

R³ and R⁴ are independently selected from branched or straight chain, substituted or unsubstituted, saturated or unsaturated alkyl, cyclic or aromatic groups including between about 1 and about 36 carbon atoms;

R⁵ and R⁶ are independently H or CH₃;

x and y are independently an integer ranging from 1 to about 10; and

Z is a counterion or salt-former.

In some embodiments, x and y are independently an integer ranging from 2 to about 8. In other embodiments, x and y are independently an integer ranging from 2 to about 5.

In some embodiments, at least one of R⁵ or R⁶ is CH₃. In other embodiments, both R⁵ and R⁶ are CH₃. In yet other embodiments, both R⁵ and R⁶ are CH₃; and x and y both range from 2 to 5. In further embodiments, both R⁵ and R⁶ are CH₃; and x and y both range from 2 to 5; R² and R² are independently methyl or ethyl; and R³ and R⁴ are independently selected from saturated or unsaturated alkyl groups having between 18 and 24 carbon atoms. In yet further embodiments, both R⁵ and R⁶ are CH₃; and x and y are 3; and R² and R² independently are methyl or ethyl; and R³ and R⁴ are independently selected from saturated or unsaturated alkyl groups having between 18 and 24 carbon atoms.

In some embodiments, the compounds of Formula (IV) have a biodegradability of at least about 50% as measured by a OECD-301B testing methodology. In other embodiments, the compounds of Formula (I) have an aquatic toxicity of at least about 10 mg/L as measured by a OECD-202: Daphnia sp. Acute Immobilization Test methodology. In yet other embodiments, the compounds of Formula (I) have a biodegradability of at least about 50% as measured by a OECD-301B testing methodology and an aquatic toxicity of at least about 10 mg/L as measured by OECD-202: Daphnia sp. Acute Immobilization Test methodology.

In some embodiments, the compounds of Formula (IV) are combined with a personal care ingredient to form a personal care composition, such as a shampoo or conditioner.

In another embodiment of the present invention, Applicants have discovered a compound of Formula (IVa):

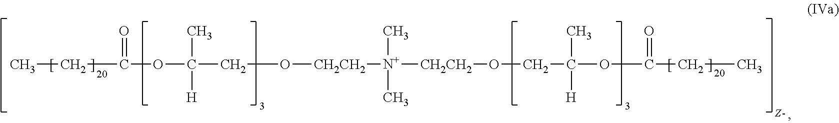

where Z is a counterion or salt-former.

In some embodiments, the diesters of Formula (IVa) are saturated. In other embodiments, the diesters of Formula (IVa) are unsaturated. In yet other embodiments, the (CH₂)₂₀ group in Formula (IVa) contains one double bond, where the double bond may have any stereochemical conformation.

In another embodiment of the present invention, Applicants have discovered the compounds of Formula (V):

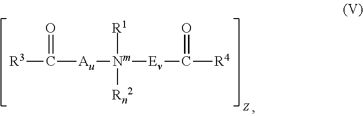

wherein R¹, R², R³, R⁴, A, E, u, v, m, n, and Z are as defined herein.

Esterquats have been available for a number of years and have predominantly been used in laundry applications. While these products are biodegradable and work well for their intended application, it is believed that they need to be formulated at pH's less than about 4.5 due to their instability at higher pH's. It has been surprisingly found that some of the compounds of the present invention overcome these limitations and can be formulated at pH's as high as 5.5-6.0 making them ideal for use in personal care products including shampoos and hair conditioners. In addition, it has been unexpectedly found that these compounds are biodegradable and/or have low aquatic toxicity values, as detailed further herein.

Accordingly, in one embodiment, the present invention is a personal care product including an amine (tertiary amine, salt or quat of the invention, and at least one personal care ingredient), having a pH (alone or when added to water) of between about 5.0 and about 7.5 and more preferably between above 5.5 and 6.0.

### DETAILED DESCRIPTION

Diester tertiary or quaternary amines in accordance with the present invention include those having the Formula (I):

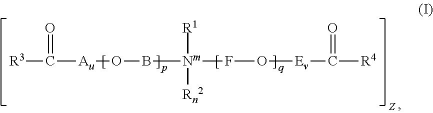

wherein R¹ and R² are independently selected from branched or straight chain, substituted or unsubstituted, saturated or unsaturated alkyl, cyclic or aromatic groups including between about 1 and about 22 carbon atoms;

B and F are independently a straight chain or branched alkyl group having between about 2 and 8 carbon atoms;

p and q are independently 0 and 1;

A and E are independently selected from straight chain or branched alkoxy groups, or mixtures thereof, having between 1 and 8 carbon atoms;

u and v may be the same or different and are each an integer greater than 1;

R³ and R⁴ are independently selected from branched or straight chain, substituted or unsubstituted, saturated or unsaturated alkyl, cyclic or aromatic groups including between about 1 and about 35 carbon atoms;

Z is a counterion or salt former;

n is 0 or 1; and

m is a lone pair of electrons or a positive charge;

with the proviso that if n is 0, then m is a lone pair of electrons and there is no counterion (Z); and if n is 1, then m is a positive charge and the counterion or salt-former (Z) is present; and

with the proviso that if Aᵤ and Eᵥ each independently represent blocks containing propoxy and ethoxy groups, the number of propoxy groups is greater than the number of ethoxy groups.

In some embodiments, the diesters of Formula (I) are tertiary amines, i.e. contain an amine group having three bonds and a neutral charge. The tertiary amines may be in the form of a salt (formed by neutralizing with any of the above acids), solvate, hydrate, or any combination thereof. In other embodiments, the diesters of Formula (I) are quaternary amines, i.e. contain an amine group having four bonds and a positive charge (referred to herein as “diester quats”). The positive charge on the quat is balanced by a counterion (Z).

Diester quats of Formula (I) have the Formula (II):

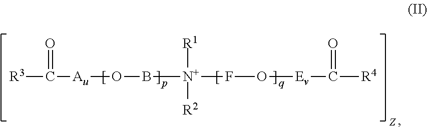

wherein R¹, R², R³, R⁴, A, E, B, F, u, v, p, q, and Z are as defined for Formula (I) above, and with the proviso that if Aᵤ and Eᵥ each independently represent blocks containing propoxy and ethoxy groups, the number of propoxy groups is greater than the number of ethoxy groups.

In some embodiments, R² and R² are independently selected from branched or straight chain, substituted or unsubstituted, saturated or unsaturated alkyl, cyclic or aromatic groups including between about 1 and about 10 carbon atoms. In other embodiments, R² and R² are independently selected from lower alkyl groups having between about 1 and about 8 carbon atoms. In yet other embodiments, R² and R² are independently selected from methyl, ethyl, n-propyl, iso-propyl, n-butyl, iso-butyl, or sec-butyl. In further embodiments, R² and R² are independently selected from methyl or ethyl. Of course, in any of the embodiments of Formula (I), n may be 0 or 1, thus providing for tertiary or quaternary amine diesters (or their salts), respectively. In yet further embodiments, n of Formula (I) is 1, m is a positive charge, and R¹ and R² are both methyl.

R³ and R⁴ are, generally, fatty acid alkyl groups or derivatives thereof. Fatty acids used in the present invention can be derived from hydrogenated or non-hydrogenated vegetable oils such as, without limitation, Palm, Coconut, Peanut, high or low Erucic Canola, Meadowfoam, Tall Oil and the like or animal oils such as beef tallow or from synthetic sources. In some embodiments, R³ and R⁴ are independently selected from saturated or unsaturated, straight chain or branched alkyl groups having between about 9 and 29 carbon atoms. In other embodiments, R³ and R⁴ are independently selected from saturated or unsaturated, straight chain or branched alkyl groups having between about 15 and 27 carbon atoms.

Alkyl groups may be saturated or unsaturated to any degree. When unsaturated, generally, the degree of saturation of any aliphatic region ranges between 1 and about 4, i.e. the aliphatic portion of the group contains between 1 and about 4 double bonds. Those skilled in the art will recognize that the unsaturated groups may have any stereochemistry, i.e. may comprise trans (E) or cis (Z) stereoisomer forms or any combination thereof.

In yet other embodiments, R³ and R⁴ are independently selected from saturated alkyl groups having between about 18 and 24 carbon atoms. In further embodiments, R³ and R⁴ are independently selected from unsaturated alkyl groups having between about 18 and about 24 carbon atoms, and having any degree of saturation.

In some embodiments, B and F are independently a straight chain or branched alkyl group having between about 2 and about 6 carbon atoms. In other embodiments, B and F are independently a straight chain or branched alkyl group having between about 2 and about 4 carbon atoms. In yet other embodiments, B and F are independently a straight chain alkyl group having between about 2 and about 4 carbon atoms. In yet further embodiments, B and F both selected from a straight chain alkyl group having two carbon atoms. In yet even further embodiments, B and F are selected from alkyl groups (straight chain or branched) having 3 to 5 carbon atoms.

As already mentioned, Z is any counterion or salt-former. In some embodiments, Z is a neutralizing agent such as a C₂-C₂₂ fatty acid, C₄-C₄₄ dimer acids, glutamic acid, and tri-acids (e.g. citric acid). Those skilled in the art will be able to select and appropriate counterion or salt-former based on the overall desired charge.

In some embodiments, u and v are independently an integer between 1 and 10. In other embodiments, u and v are independently an integer between 2 and 8. In yet other embodiments, u and v are independently an integer between 2 and 6. In yet further embodiments, u and v are independently the integer 3. In yet even further embodiments, u and v are independently the integer 5.

In some embodiments, A and E are straight chain or branched alkoxy groups having between about 1 and 6 carbon atoms. In other embodiments, A and E are straight chain or branched alkoxy groups having between about 2 and about 4 carbon atoms. In yet other embodiments, A and E are ethoxy or propoxy groups (as used herein, the term “propoxy” refers to linear or branched alkoxy groups containing three carbon atoms).

As detailed further below, any of A or E may represent a mixture of alkoxy groups. That said, Aᵤ and Eᵥ may independently represent blocks of alkoxy groups, where the “blocks” may be the same or mixed. By way of example, A or E independently may be a combination of 3 ethoxy groups and 2 propoxy groups as in the following formula: —[(O—CH₂CH₂) 3]-[(O—CH(CH₃)CH₂)₂]. Thus, as in the foregoing example, the invention contemplates mixed blocks of straight chain or branched alkoxy groups, where each alkoxy group has between about 1 and 6 carbon atoms. Mixed blocks may be in the form of XX-YY blocks where XX and YY represent different alkoxy groups. Mixed blocks may also be in the form of XY-XY blocks, where X and Y represent different alkoxy groups. Of course, the blocks may contain a mixture of more than two different alkoxy groups and may have any repeat group (repetitive pattern).

In some embodiments, Aᵤ and Eᵥ of Formulas (I) or (II) are independently selected from the group having the Formula (III):

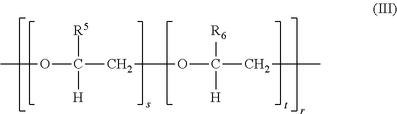

wherein R⁵ and R⁶ are independently H or CH₃;

s and t are independently 0 or an integer ranging between 1 and 10; and

r is an integer greater than 1.

According to Formula (III), the present invention may comprise a number of repeating ethoxy and/or propoxy groups (or blocks), in any order. Not illustrated, but still contemplated by the present invention, are embodiments where the propoxy group of Formula (III), if present when R⁵ or R⁶ is carbon, is a straight-chain (i.e. —O—CH₂—CH₂—CH₂—). Thus, to be clear, Formula (III) contemplates straight chain and/or branched propoxy groups.

In some embodiments, the diesters of the present invention comprise both propoxy and ethoxy groups. In other embodiments, the diesters of the present invention comprise only propoxy groups (straight chain or branched) or only ethoxy groups. In yet other embodiments, the number of propoxy groups in any one diester are greater than the number of ethoxy groups contained therein. In yet further embodiments, a ratio of the number of propoxy groups to the number of ethoxy groups ranges from about 3:2 to about 2:3.

In some embodiments, one of s or t is 0. In other embodiments, one of R⁵ or R⁶ is CH₃ and the other of R⁵ or R⁶ is H. In yet other embodiments, one of R⁵ or R⁶ is CH₃ and the other of R⁵ or R⁶ is H; one of s or t is 2 and the other of s or t is 3; and r is 1. In further embodiments, Formula (III) represents both Aᵤ and Eᵥ; one of R⁵ or R⁶ is CH₃ and the other of R⁵ or R⁶ is H; one of s or t is 2 and the other of s or t is 3; and r is 1.

By way of example, one compound where Aᵤ and Eᵥ are represented by Formula (III) is in Formula (IIIa):

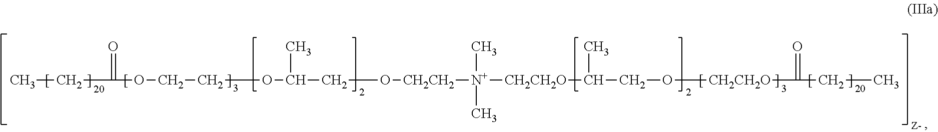

In another embodiment, the compounds of Formula (IIIa) comprise linear propoxy groups, wherein Z is a counterion or salt former as defined above.

The alkyl groups of the compounds of Formula (IIIa) may be saturated or unsaturated to any degree. In some embodiments, the diesters of Formula (IIIa) are saturated. For example, the groups (CH₂)₂₀ may be completely saturated. In other embodiments, the diesters of Formula (IIIa) are unsaturated. For example, the groups (CH₂)₂₀ may contain at least one degree of saturation, i.e. at least one double bond, and may be in any stereoisomeric form (cis or trans). In yet other embodiments, the (CH₂)₂₀ group in Formula (IIIa) contains one double bond.

In some embodiments, the diesters of Formula (I) or Formula (II) have the structure of Formula (IV)

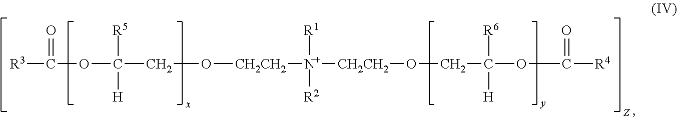

wherein R¹, R², R³, R⁴, and Z are as defined above;

R⁵ and R⁶ are independently H or CH₃;

x and y are independently an integer ranging from 1 to 10;

and Z is a counterion or salt-former.

In some embodiments, x and y are independently an integer ranging from 1 to about 8. In other embodiments, x and y are independently an integer ranging from 2 to about 8. In yet other embodiments, x and y are independently an integer ranging from 2 to about 6. In further embodiments, x and y are independently an integer ranging from 2 to 5. In yet further embodiments, x and y are independently the integer 3. In yet even further embodiments, x and y are independently the integer 5.

In some embodiments, at least one of R⁵ or R⁶ is CH₃. In other embodiments, both R⁵ and R⁶ are CH₃. In yet other embodiments, both R⁵ and R⁶ are CH₃; and x and y both range from 2 to 5. In further embodiments, R⁵ and R⁶ are both CH₃; and x and y are both 3.

Examples of diesters of Formula (IV) include those of Formula (IVa):

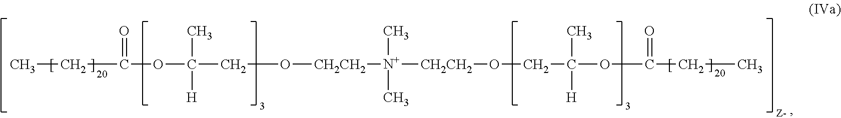

wherein Z is a counterion or salt former as defined above. Not illustrated, but contemplated, are compounds similar to Formula (IVa) which contain a straight-chain propoxy group.

Other examples of diesters of Formula (IV) include those of Formula (IVb):

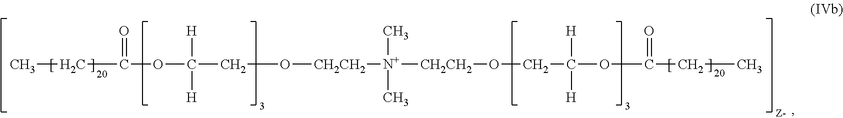

wherein Z is a counterion or salft former as defined above.

In some embodiments, the diesters of Formulas (IVa) or (IVb) are saturated. For example, the groups (CH₂)₂₀ may be completely saturated. In other embodiments, the diesters of Formulas (IVa) or (IVb) are unsaturated. For example, the groups (CH₂)₂₀ may contain at least one degree of saturation, i.e. contain at least one double bond, and may be in any stereoisomeric form (cis or trans). In yet other embodiments, the (CH₂)₂₀ group in Formulas (IVa) or (IVb) contain one double bond.

Another example of a diester of Formula (IV) includes the compound of Formula (IVc):

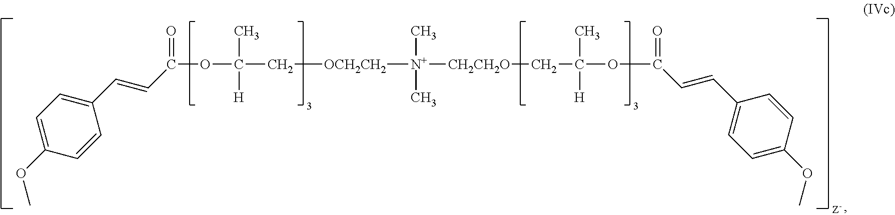

wherein Z is a counterion or salt former as defined above, and where the double bonds independently may have any stereo conformation, i.e. cis or trans.

In some embodiments, the diesters of Formula (I) or Formula (II) have the structure of Formula (V)

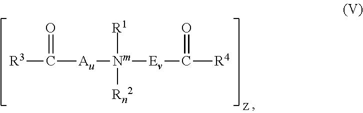

wherein R¹, R², R³, R⁴, A, E, u, v, m, n, and Z are as defined herein. As with Formulas (I) and (II), Aᵤ and Eᵥ of Formula (V) may be represented by Formula (III). In some embodiments, n is 0 and m is a lone pair of electrons (tertiary amine). In other embodiments, n is 1 and m is a positive charge (quaternary amine).

In some embodiments, A and E of Formula (V) are independently selected from ethoxy and propoxy (straight chain or branched). In other embodiments, A and E are both ethoxy. Of course, as described herein, Aᵤ and Eᵥ may represent blocks of ethoxy and propoxy groups or may be represented by Formula (III). In yet other embodiments, A and E are both ethoxy; u and v are independently an integer between 2 and 5; R³ and R⁴ independently represent aliphatic carbon chains (straight chain or branched, substituted or unsubstituted) having between about 18 and about 24 carbon atoms; and R¹ and R² independently are methyl or an aliphatic carbon chain (straight chain or branched, substituted or unsubstituted) having between about 10 and about 20 carbon atoms.

In some embodiments, the compounds of the present invention are biodegradable as provided by standardized OECD 301B testing. The testing methodology is described in the “OECD Guideline for Testing Chemicals,” section 301, the disclosure of which is incorporated by reference. Specifically, the compounds of the present invention showing a biodegradability of at least about 50% according to the OECD 301B testing method. In some embodiments, the compounds of the present invention have a biodegradability of between about 50% to about 81% according to the OECD 301B testing method.

In some embodiments, the compounds of the present invention are believed to have low aquatic toxicities. Specifically, the compounds of the present invention have EC₅₀ (24 h) levels of at least about 10 mg/L according to OECD 202 testing methodology. In some embodiments, the compounds of the present invention have EC₅₀ (24 h) levels of between about 5 mg/L and about >100 mg/L according to OECD 202 testing methodology. The testing methodology is described in “OECD Guideline for Testing of Chemicals,” section 202, the disclosure of which is incorporated by reference.

In some embodiments, the compounds of the present invention meet both the biodegradability and aquatic toxicity standards outlined above.

In some embodiments, the diesters of the present invention, alone or when mixed with a solvent, additive or other compound, have a pH ranging between about 4 and about 8, preferably having a pH ranging between about 5 and about 7.

The diesters of the present invention can be synthesized as described in U.S. Pat. No. 7,202,204, the disclosure of which is incorporated by reference herein.

Generally, the diesters of the invention may be synthesized by well known methods. Condensation reactions are predominant.

For example, an alkyl diethanol amine can be reacted with various proportions of alkoxylating compounds to produce alkoxylated amine species. The amount and order of addition of these alkoxylating species will determine their relative arrangement and proportion. For example, if in Step 1AA, M moles is 10, then the resulting compound will be symmetrical and with u and v both being 5 and A and E being five consecutive ethoxy groups each. If the next reactant added is N Moles, which is 2, then the sixth alkoxy group for each side will be propoxy. It will be appreciated that variations can occur and therefore it is possible that the result will be a mixture that is neither symmetric nor in the desired order. However, generally, the predominant fraction of all of the alkoxylated amines produced will be the desired structure. These can be separated to isolate the amine of the desired structure. The reaction can proceed as illustrated in 1AA.

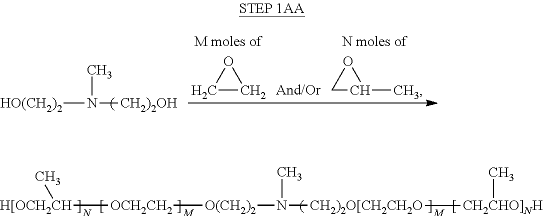

The alkoxylated material of Step IAA can then be reacted with one of the groups disclosed herein for R³ or R⁴ (e.g., a fatty acid derivate).

Alternatively, it may be desirable to produce various chains of alkoxy groups separately and then react them either with a fatty acid, fatty alcohol, an oil derivate, glyceride or an alkyl diethanol amine. This allows one to tailor the specific chain length and order of the alkoxy groups more precisely. Indeed, separations can be performed to eliminate mixtures as the chain length grows. Alternatively, a fatty acid or fatty alcohol can be alkoxylated (in the case of a fatty alcohol, a bridging diacid or triacid may be necessary) and the resulting molecule then reacted with the alkyl diethanol amine to produce compounds like those found in Formula (I). However, obviously, these compounds are tertiary amines until they have been quaternized.

Lastly, quats may be formed. This is done after the diester tertiary amines are completely formed. After the fatty acids, for example, are reacted with the alkoxylated amine, the resulting diester tertiary amines are then quaternized using known techniques with any suitable alkylating agent that can provide the appropriate R¹ or R² group. These can include methyl chloride, ethyl chloride, benzyl chloride, behyneal halide, dimethyl sulfate, diethyl sulfate, etc. One skilled in the art will be able to select a reagent which can provide the desired R¹ or R² group.

The diester compositions may be blended with, for example, alkylamidopropyl dimethylamines, such as Incromine BD supplied by Croda Inc., to yield a waxy flakeable or pastillatable composition with improved high temperature warehouse storage stability. Stability in this case being defined as resistance of the waxy particles from fusing together at elevated warehouse storage temperatures of about 95° F. Additional information on flakeability/pastillatability can be found in U.S. Pat. No. 6,638,497, the disclosure of which is hereby incorporated by reference herein.

In some embodiments, the diesters of the claimed invention are blended with another tertiary amine, which, in preferred embodiments is itself (1) biodegradable and/or non-toxic, and (2) will not hinder the biodegradability of the compounds of Formulas (I)-(IV) and/or cause toxicity. This blend can also be incorporated into the personal care formulations described herein. Suitable tertiary amines include amindoamines, such as those disclosed in U.S. Pat. Nos. 6,365,142 and 4,891,214, the disclosures of which are hereby incorporated by reference herein. Particularly suitable tertiary amines include Behenamidopropyldimethylamine and Erucamidopropyldimethylamine.

In some embodiments, it is desirable that the diesters of the claimed invention be either liquids or waxy solids in pastille form at room temperature for ease of use. Applicants have discovered that some waxy solid pastillated diesters did not have good storage stability at 95 F and ended up fusing together. By adding behenamidopropydimethylamine, the high temperature storage stability of the diester was increased, allowing for it to be warehoused or transported in conditions were the temperature can exceed 95° F. without the pastilles fusing together.

### Personal Care Compositions

The invention provides personal care compositions including one or more of the diester compounds (or salts, solvates, or hydrates thereof) of Formula (I) (or of any other Formula recited herein) and at least one personal care ingredient. The composition may also comprise one or more optional solvents.

Thus, in one embodiment of the present, Applicants have discovered a personal care product comprising a compound of Formula (I) and a personal care ingredient:

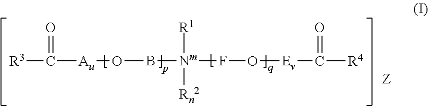

wherein R¹ and R² are independently selected from branched or straight chain, substituted or unsubstituted, saturated or unsaturated alkyl, cyclic or aromatic groups including between about 1 and about 22 carbon atoms;

B and F are independently a straight chain or branched alkyl group having between 2 and 8 carbon atoms,

p and q are independently 0 or 1;

A and E are independently selected from a straight chain or branched alkoxy group having between 1 and 8 carbon atoms;

u and v may be the same or different and are each an integer greater than 1;

R³ and R⁴ are independently selected from branched or straight chain, substituted or unsubstituted, saturated or unsaturated alkyl, cyclic or aromatic groups including between about 1 and about 36 carbon atoms;

Z is a counterion or salt former and included only in cases where the compounds of Formula (I) are a salt or quat as appropriate (hereinafter “counterion”);

n is 0 or 1;

m is a lone pair of electrons or a positive charge; and

with the proviso that if n is 0, then m is a lone pair of electrons and there is no counterion (Z); and if n is 1, then m is a positive charge and the counterion (Z) is present.

The diester compound to be used in the personal care compositions depends on the type of product and its intended use. One or more of the diesters of Formulas I, II, III, IV, or V, or can be used in any personal care product. The diesters of Formula I, II, III, IV, or V, may also be mixed with other quaternary or tertiary amines known in the art, as disclosed herein.

In accordance with the invention, the personal care compositions or products contemplated herein include, without limitation, cosmetics, sunscreen compositions, lotions, hand cleaners, bath compositions, suntan oils, antiperspirant compositions, perfumes and colognes, cold creams, pre-shaves, deodorants, pharmaceutical preparations, skin moisturizers, facial cleansers, cleansing creams, skin gels, shampoos, hair conditioners, rinses, cream rinses, detergents, make-up products, permanent waving products, lipsticks, mascara, blush, foundation, rouge, mousse, sprays, styling gels, nail care products, and dyes and hair coloring products.

The amount of the diester quats in accordance with the invention found in the resulting personal care product generally range from about 0.1 to about 20% on a cationic activity basis. More preferably, the amount is between about 0.5 to about 15%, more preferably about 1% to about 10% on a cationic activity basis. Most preferred, the amount of diester quats of the invention in personal care products will range from between about 1 to about 5% based on a cationic activity basis.

Cationic activity may be measured by several methods readily understood by those skilled in the art. One such method utilizes a standardized solution of an anionic material, such as sodium lauryl sulfate. This material is added to the solution containing the quat until full complexation of the quat's cations (the end point) has been reached. The end point can be measured potentiometrically or by the use of color indicators.

Typical tests involve titrating a sample of the quat, usually dissolved in a solvent, with the standardized solution of sodium lauryl sulfate until the endpoint is reached. As described in U.S. patent application Ser. No. 09/438,631, incorporated by reference herein in its entirety, once the endpoint is reached, the cationic activity can be calculated according to the following formula:

$$\% cationic activity = \frac{mL \times N \times MW \times 100}{S . wt . \times 1000}$$

where:

mL=the number of mL of anionic material;

N=the normality of the solution used;

MW=the equivalent molecular weight of the quat being analyzed; and

S.wt.=the sample weight in grams.

For additional information regarding the methodology for measuring the cationic activity, see W. Schempp and H. T. Trau, Wochenblatt fur Papierfabrikation 19, 1981, pp. 726-732, or J. P. Fischer and K. Lohr, Organic Coatings Science Technology, Volume 8, pp. 227-249, Marcel Dekker, Inc. April 1986), both incorporated herein by reference in their entirety. The personal care products may be in the form of liquids, ointments, lotions, sprays, gels, creams, emulsions, foams, pastes and solids; may be clear or opaque; and may be formulated as aqueous and non-aqueous preparations.

### Solvents

In some embodiments, such final products are dispersions or solutions in water, or in a mixture of water with a suitable secondary solvent. Suitable solvents include various lower alkanols and glycols having from 1 to 8 carbon atoms, including methanol, ethanol, isopropanol, butanol, hexylene glycol, 1,3-butylene glycol, 1,2- and 1,3-propane diol, 2-methyl 1,3-propane diol, propylene glycol, diethylene glycol, and the like.

The total amount of solvent, including water and mixtures of water and solvents, may be up to about 98% by weight of the composition; in some embodiments from about 20% to about 90% by weight of the composition and; in other embodiments from about 50% to about 90% by weight of the composition.

In other embodiments, final products are waxy solid mixtures in alkylamidoamines such as Behenamidopropyldimethylamine or fatty alcohol such as cetyl, steary or behenyl alcohol or mixtures thereof.

The total amount of solvent may be up to about 90% by weight of the composition; in some embodiments from about 5% to about 85% by weight of the composition and; in other embodiments from about 10% to about 80% by weight of the composition.

### Personal Care Ingredients

Personal care ingredients include, without limitation, cleaning agents, hair conditioning agents, skin conditioning agents, hair styling agents, antidandruff agents, hair growth promoters, perfumes, sunscreen compounds, pigments, moisturizers, film formers, humectants, alpha-hydroxy acids, hair colors, make-up agents, detergents, thickening agents, emulsifiers, antiseptic agents, deodorant actives and surfactants. They may also include agents which enhance permeation into or through the skin, or topical pharmaceuticals such as, without limitation, corticosteriods, analgesics, anti-inflammatory agents, antibiotics, anesthetics, etc. These may all be used in conventional and/or approved amounts.

Personal care ingredients can be used in any form, including in a liquid or solid form. Solids can be crystalline or amorphous, granular, powder, particulate and the like. It is also possible for such ingredients to be microencapsulated or in the form of micro-particles.

### Surfactants

One type of personal care ingredient which may be used are surfactants including nonionic surfactants, anionic surfactants, cationic surfactants, amphoteric surfactants, zwitterionic surfactants, and mixtures thereof.

### Cationic Surfactants

Cationic surfactants suitable for use in personal care products include quaternary ammonium cationic surfactants of the formula [N⁺(Q₄)(Q₂)(Q₃)(Q₄)]_{d}Z, Q₁ is C₁₂-C₂₂ alkyl, C₁₂-C₂₂ alkyl amido, C₁-C₆ alkylene, C₁₂-C₂₂ alkylhydroxy; Q₂ is C₁₂-C₂₂ alkyl, C₁₂-C₂₂ alkyl amido C₁-C₆ alkylene, C₁₂-C₂₂ alkylhydroxy, benzyl, or C₁-C₆ alkyl; Q₃ and Q₄ are independently C₁-C₆ alkyl or benzyl; Z is a counterion; and d is an integer greater than 0.

Examples of quaternary ammonium cationic surfactants include cetyl ammonium chloride, cetyl ammonium bromide, lauryl ammonium chloride, lauryl ammonium bromide, stearyl ammonium chloride, stearyl ammonium bromide, cetyl dimethyl ammonium chloride, cetyl dimethyl ammonium bromide, lauryl dimethyl ammonium chloride, lauryl dimethyl ammonium bromide, stearyl dimethyl ammonium chloride, stearyl dimethyl ammonium bromide, cetyl trimethyl ammonium chloride, cetyl trimethyl ammonium bromide, lauryl trimethyl ammonium chloride, lauryl trimethyl ammonium bromide, stearyl trimethyl ammonium chloride, stearyl trimethyl ammonium bromide, lauryl dimethyl ammonium chloride, stearyl dimethyl cetyl ditallow dimethyl ammonium chloride, dicetyl ammonium chloride, dicetyl ammonium bromide, dilauryl ammonium chloride, dilauryl ammonium bromide, distearyl ammonium chloride, distearyl ammonium bromide, dicetyl methyl ammonium chloride, dicetyl methyl ammonium bromide, dilauryl methyl ammonium chloride, dilauryl methyl ammonium bromide, distearyl methyl ammonium chloride, distearyl dimethyl ammonium chloride, distearyl methyl ammonium bromide, and mixtures thereof.

Additional quaternary ammonium cationic surfactants include those wherein the C₁₂-C₂₂ alkyl is derived from a tallow fatty acid or from a coconut fatty acid, including ditallow dimethyl ammonium chloride, ditallow dimethyl ammonium methyl sulfate, di(hydrogenated tallow) dimethyl ammonium chloride, di(hydrogenated tallow) dimethyl ammonium acetate, ditallow dipropyl ammonium phosphate, ditallow dimethyl ammonium nitrate, di(coconut alkyl)dimethyl ammonium chloride, di(coconut alkyl)dimethyl ammonium bromide, tallow ammonium chloride, coconut ammonium chloride, stearamidopropyl PG-dimonium chloride phosphate, stearamidopropyl ethyldimonium ethosulfate, stearamidopropyl dimethyl (myristyl acetate) ammonium chloride, stearamidopropyl dimethyl cetearyl ammonium tosylate, stearamidopropyl dimethyl ammonium chloride, stearamidopropyl dimethyl ammonium lactate, and mixtures thereof.

In some embodiments, the quaternary ammonium cationic surfactants are dilauryl dimethyl ammonium chloride, distearyl dimethyl ammonium chloride, dimyristyl dimethyl ammonium chloride, dipalmityl dimethyl ammonium chloride, distearyl dimethyl ammonium chloride, stearamidopropyl PG-dimonium chloride phosphate, stearamidopropyl ethyldimonium ethosulfate, stearamidopropyl dimethyl (myristyl acetate) ammonium chloride, stearamidopropyl dimethyl cetearyl ammonium tosylate, stearamidopropyl dimethyl ammonium chloride, stearamidopropyl dimethyl ammonium lactate, and mixtures thereof.

The total amount of cationic surfactants may range from about 0.1% to about 40%, more preferably from about 0.1% to about 15%, yet more preferably, from about 0.5% to about 2%, by the weight of the product composition.

### Non-Ionic Surfactants

The compositions of the invention may include non-ionic surfactants, including the condensation products of C₈-C₃₀ alcohols with sugar or starch polymers. These compounds can be represented by the formula (S)ₙ—O—R, wherein S is a sugar moiety such as glucose, fructose, mannose, and galactose; n is an integer of from about 1 to about 1000, and R is C₈-C₃₀ alkyl. Examples of suitable C₈-C₃₀ alcohols from which the R group may be derived include decyl alcohol, cetyl alcohol, stearyl alcohol, lauryl alcohol, myristyl alcohol, oleyl alcohol, and the like. Specific examples of these surfactants include decyl polyglucoside and lauryl polyglucoside.

Other non-ionic surfactants include the condensation products of alkylene oxides with fatty acids, having the formula RCO(X)ₙOH, wherein R is a C₁₀-C₃₀ alkyl, X is —OCH₂CH₂— (derived from ethylene oxide) or —OCH₂CHCH₃— (derived from propylene oxide), and n is an integer from about 1 to about 200.

Yet other non-ionic surfactants are the condensation products of alkylene oxides with fatty acids having the formula RCO(X)ₙOOCR, wherein R is a C₁₀-C₃₀ alkyl, X is —OCH₂CH₂— (derived from ethylene oxide) or —OCH₂CHCH₃— (derived from propylene oxide), and n is an integer from about 1 to about 200. Yet further non-ionic surfactants are the condensation products of alkylene oxides with fatty alcohols having the general formula R(X)ₙOR′, wherein R is C₁₀-C₃₀ alkyl, n is an integer from about 1 to about 200, and R′ is H or a C₁₀-C₃₀ alkyl.

Still other non-ionic surfactants are the compounds having the formula RCO(X)ₙOR′, wherein R and R′ are C₁₀-C₃₀ alkyl, X is —OCH₂CH₂— (derived from ethylene oxide, an alkylene oxide) or —OCH₂CHCH₃— (derived from propylene oxide, an alkylene oxide), and n is an integer from about 1 to about 200.

Examples of alkylene oxide-derived non-ionic surfactants include ceteth-1, ceteth-2, ceteth-6, ceteth-10, ceteth-12, ceteraeth-2, ceteareth-6, ceteareth-10, ceteareth-12, steareth-1, steareth-2, stearteth-6, steareth-10, steareth-12, PEG-2 stearate, PEG-4 stearate, PEG-6 stearate, PEG-10 stearate, PEG-12 stearate, PEG-20 glyceryl stearate, PEG-80 glyceryl tallowate, PPG-10 glyceryl stearate, PEG-30 glyceryl cocoate, PEG-80 glyceryl cocoate, PEG-200 glyceryl tallowate, PEG-8 dilaurate, PEG-10 distearate, and mixtures thereof.

Still other useful non-ionic surfactants include polyhydroxy fatty acid amides disclosed, for example, in U.S. Pat. Nos. 2,965,576, 2,703,798, and 1,985,424, the disclosures of which are incorporated herein by reference.

Non-ionic surfactants may be present in amounts ranging from about 0.1% to about 40%, more preferably, from about 0.1% to about 15%, and yet more preferably, from about 0.5% to about 2%, by weight of the final formulation.

### Anionic Surfactants

The compositions of the invention may include anionic surfactants, examples of which are disclosed in U.S. Pat. No. 3,929,678, which is incorporated herein by reference. Further examples of suitable anionic surfactants include alkoyl isethionates, and alkyl ether sulfates.

The alkoyl isothionates typically have the formula RC(O)OCH₂CH₂—SO₃M, wherein R is C₁₀-C₃₀ alkyl, and M is a water-soluble cation, such as ammonium, sodium, potassium, or triethanolamine. The examples of suitable isethionates include ammonium cocoyl isethionate, sodium cocoyl isethionate, sodium lauroyl isethionate, sodium stearoyl isethionate, and mixtures thereof. Preferred for used herein are ammonium cocoyl isethionate, sodium cocoyl isethionate, and mixtures thereof.

The alkyl ether sulfates typically have the formulas ROSO₂M and RO(C₂H₄O)ₓSO₂M, where R is C₁₀-C₃₀ alkyl, x varies from about 1 to about 10, and M is a water-soluble cation such as ammonium, sodium, potassium and triethanolamine.

Yet another suitable class of anionic surfactants are alkali metal salts of C₈-C₃₀ carboxylic acids and alkylsulfonates of the formula R₁—SO₃M (where R₁ is C₈-C₃₀ alkyl; preferably, C₁₂-C₂₂ alkyl, and M is a cation), including succinamates, and C₁₂-C₂₄ olefin sulfonates and carboxylates.

Anionic surfactants may be present in amounts ranging from about 0.1% to about 40%, more preferably, from about 0.1% to about 15%, and yet more preferably, from about 0.5% to about 2%, by weight of the formulation.

### Zwitterionic and Amphoteric Surfactants

The compositions of the invention may include zwitterionic and amphoteric surfactants, such as derivatives of mono- or di-C₈-C₂₄ secondary and tertiary amines, such as alkyl imino acetates, carboxylates, sulfonates, sulfates, phosphates, and phosphonates, including iminodialkanoates and aminoalkanoates of the formulas RN(CH₂)ₘCO₂M₂ and RNH(CH₂)ₘCO₂M, where m varies from 1 to 4, R is C₈-C₃₀ alkyl; preferably, C₁₂-C₂₂ alkyl, and M is H, alkali metal, alkaline earth metal ammonium, or alkanolammonium.

Other suitable amphoteric and zwitterionic surfactants are imidazolinium and ammonium derivates, including sodium 3-dodecyl-aminopropionate, sodium 3-dodecylaminopropane sulfonate, N-alkyltaurines; N-higher alkyl aspartic acids, and coamidopropyl PG-dimonium chloride phosphate. For further examples of suitable amphoteric and zwitterionic surfactants, please see U.S. Pat. Nos. 2,658,072, 2,438,091, and 2,528,378, which are incorporated herein by reference.

Yet other suitable amphoteric and zwitterionic surfactants are betaines, including coco dimethyl carboxymethyl betaine, lauryl dimethyl carboxymethyl betaine, lauryl dimethyl alphacarboxyethyl betaine, cetyl dimethyl carboxymethyl betaine, cetyl dimethyl betaine, lauryl bis-(2-hydroxyethyl) carboxymethyl betaine, stearyl bis-(2-hydroxypropyl) carboxymethyl betaine, oleyl dimethyl gamma-carboxypropyl betaine, lauryl bis-(2-hydroxypropyl) alpha-carboxyethyl betaine, coco dimethyl sulfopropyl betaine, stearyl dimethyl sulfopropyl betaine, lauryl dimethyl sulfoethyl betaine, lauryl bis-(2-hydroxyethyl) sulfopropyl betaine, and amidobetaines and amidosulfobetaines, oleyl betaine, and cocamidopropyl betaine.

### Other Quaternary Ammonium Compounds

The compositions of the invention may include quaternary ammonium compounds or salts, other then those of Formula (I) or Formula (II).

Examples of suitable quaternary ammonium compounds include cetyl ammonium chloride, cetyl ammonium bromide, lauryl ammonium chloride, lauryl ammonium bromide, stearyl ammonium chloride, stearyl ammonium bromide, cetyl dimethyl ammonium chloride, cetyl dimethyl ammonium bromide, lauryl dimethyl ammonium chloride, lauryl dimethyl ammonium bromide, stearyl dimethyl ammonium chloride, stearyl dimethyl ammonium bromide, cetyl trimethyl ammonium chloride, cetyl trimethyl ammonium bromide, lauryl trimethyl ammonium chloride, lauryl trimethyl ammonium bromide, stearyl trimethyl ammonium chloride, stearyl trimethyl ammonium bromide, lauryl dimethyl ammonium chloride, stearyl dimethyl cetyl ditallow dimethyl ammonium chloride, dicetyl ammonium chloride, dicetyl ammonium bromide, dilauryl ammonium chloride, dilauryl ammonium bromide, distearyl ammonium chloride, distearyl ammonium bromide, dicetyl methyl ammonium chloride, dicetyl methyl ammonium bromide, dilauryl methyl ammonium chloride, dilauryl methyl ammonium bromide, distearyl methyl ammonium chloride, distearyl dimethyl ammonium chloride, distearyl methyl ammonium bromide, and mixtures thereof.

Additional quaternary ammonium salts include those wherein the C₁₂-C₂₂ alkyl is derived from a tallow fatty acid or from a coconut fatty acid. Examples of quaternary ammonium salts derived from these tallow and coconut sources include ditallow dimethyl ammonium chloride, ditallow dimethyl ammonium methyl sulfate, di(hydrogenated tallow) dimethyl ammonium chloride, di(hydrogenated tallow) dimethyl ammonium acetate, ditallow dipropyl ammonium phosphate, ditallow dimethyl ammonium nitrate, di(coconut alkyl)dimethyl ammonium chloride, di(coconut alkyl)dimethyl ammonium bromide, tallow ammonium chloride, coconut ammonium chloride, stearamidopropyl PG-dimonium chloride phosphate, stearamidopropyl ethyldimonium ethosulfate, stearamidopropyl dimethyl (myristyl acetate) ammonium chloride, stearamidopropyl dimethyl cetearyl ammonium tosylate, stearamidopropyl dimethyl ammonium chloride, stearamidopropyl dimethyl ammonium lactate, and mixtures thereof.

In some embodiments, the quaternary ammonium compounds are dilauryl dimethyl ammonium chloride, distearyl dimethyl ammonium chloride, dimyristyl dimethyl ammonium chloride, dipalmityl dimethyl ammonium chloride, distearyl dimethyl ammonium chloride, stearamidopropyl PG-dimonium chloride phosphate, stearamidopropyl ethyldimonium ethosulfate, stearamidopropyl dimethyl (myristyl acetate) ammonium chloride, stearamidopropyl dimethyl cetearyl ammonium tosylate, stearamidopropyl dimethyl ammonium chloride, stearamidopropyl dimethyl ammonium lactate, and mixtures thereof.

When present, quaternary ammonium compounds (other than those of the diester compounds of Formula (I) or Formula (II)) can be provided in any amount desirable, with the amount depending upon the purpose of the end formulation and its overall composition. Generally, these quaternary ammonium compositions may be present in an amount of between about 0 and about 50% by weight of the personal care composition.

### Sunscreens

A wide variety of sunscreen compounds (e.g. UV blocking substances) are suitable for use with the compositions of the present invention and may be present in an amount of up to about 40% by weight of the composition.

Sunscreens may be in the form of shampoos, conditioners including so-called “leave-in” conditioners, hairsprays, as well as products specifically intended as sunscreens for hair and/or skin including lotions, gels, sprays and the like.

Suitable sunscreen compounds include p-aminobenzoic acid, its salts and its derivatives; anthranilates; salicylates; cinnamic acid derivatives; dihydroxycinnamic acid derivatives; trihydroxycinnamic acid derivatives; hydrocarbons; dibenzalacetone and benzalacetophenone; naphtholsulfonates; dihydroxy-naphtholic acid and its salts; coumarin derivatives; diazoles; quinine salts; quinoline derivatives; hydroxy- or methoxy-substituted benzophenones; uric and vilouric acids; tannic acid and its derivatives; hydroquinone; amino benzoates, salicylates, ferrulic acid derivatives, phenylbenzimidazole sulfonic acids, benzophenone sulfonic acids, thioctic acids derivatives, oil-soluble cinnamates, and benzophenones.

Specific sunscreen compounds include 2-ethylhexyl p-methoxycinnamate, 4,4′-t-butyl methoxydibenzoylmethane, 2-hydroxy-4-methoxybenzophenone, octyldimethyl p-aminobenzoic acid, digalloyltrioleate, 2,2-dihydroxy-4-methoxybenzophenone, ethyl-4->bis(hydroxypropyl)-aminobenzoate, 2-ethylhexyl-2-cyano-3,3-diphenylacrylate, 2-ethylhexylsalicylate, glyceryl p-aminobenzoate, 3,3,5-trimethylcyclohexylsalicylate, methylanthranilate, p-dimethyl-aminobenzoic acid or aminobenzoate, 2-ethylhexyl p-dimethylaminobenzoate, 2-phenylbenzimidazole-5-sulfonic acid, 2-(p-dimethyl-aminophenyl)-5-sulfonicbenzoxazoic acid, para-aminobenzoic acid, benzophenone-1, benzophenone-1, benzophenone-2, benzophenone-3, benzophenone-4, benzophenone-5, benzophenone-6, benzophenone-8, benzophenone-12, methoxycinnamate, avobenzone, ethyl dihydroxypropyl para-aminobenzoate, glyceryl para-aminobenzoate, methyl anthranilate, octocrylene, octyl dimethyl para-aminobenzoate, octyl methoxycinnamate, octyl salicylate, zinc oxide, titanium dioxide, and red petrolatum.

### Emollients

The personal care compositions of the invention may include one or emollient compounds such as fats, waxes, lipids, silicones, hydrocarbons, and fatty alcohols. Emollients are included in the amount of up to about 50% by weight of the composition, preferably, from about 0.1% to about 20%, and more preferably, from about 0.5% to about 10%, by weight of the composition.

Examples of emollients include C₈₋₃₀ alkyl esters of C₈₋₃₀ carboxylic acids; lipids, C₁₋₆ diol monoesters and diesters of C₈₋₃₀ carboxylic acids; monoglycerides, diglycerides, and triglycerides of C₈₋₃₀ carboxylic acids, cholesterol esters of C₈₋₃₀ carboxylic acids, cholesterol, and hydrocarbons. Examples of these materials include diisopropyl adipate, isopropyl myristate, isopropyl palmitate, ethylhexyl palmitate, isodecyl neopentanoate, C₁₂₋₁₅ alcohols benzoates, diethylhexyl maleate, PPG-14 butyl ether, PPG-2 myristyl ether propionate, cetyl ricinoleate, cholesterol stearate, cholesterol isosterate, cholesterol acetate, jojoba oil, cocoa butter, Shea butter, lanolin, lanolin esters, mineral oil, petrolatum, and straight and branched C₁₆-C₃₀ hydrocarbons.

Other emollients useful are straight and branched chain fatty C₈-C₃₀ alcohols, for example, stearyl alcohol, isostearyl alcohol, ethenyl alcohol, cetyl alcohol, isocetyl alcohol, and mixtures thereof. Examples of other suitable emollients are disclosed in U.S. Pat. No. 4,919,934; which is incorporated herein by reference in its entirety.

Other suitable emollients are various alkoxylated ethers, diethers, esters, diesters, and trimesters. Examples of suitable alkoxylated ethers include PPG-10 butyl ether, PPG-11 butyl ether, PPG-12 butyl ether, PPG-13 butyl ether, PPG-14 butyl ether, PPG-15 butyl ether, PPG-16 butyl ether, PPG-17 butyl ether, PPG-18 butyl ether, PPG-19 butyl ether, PPG-20 butyl ether, PPG-22 butyl ether, PPG-24 butyl ether, PPG-30 butyl ether, PPG-11 stearyl ether, PPG-15 stearyl ether, PPG-10 oleyl ether, PPG-7 lauryl ether, PPG-30 isocetyl ether, PPG-10 glyceryl ether, PPG-15 glyceryl ether, PPG-10 butyleneglycol ether, PPG-15 butylene glycol ether, PPG-27 glyceryl ether, PPG-30 cetyl ether, PPG-28 cetyl ether, PPG-10 cetyl ether, PPG-10 hexylene glycol ether, PPG-15 hexylene glycol ether, PPG-10 1,2,6-hexanetriol ether, PPG-15 1,2,6-hexanetriol ether, and mixtures thereof.

Examples of alkoxylated diethers include PPG-10 1,4-butanediol diether, PPG-12 1,4-butanediol diether, PPG-14 1,4-butanediol diether, PPG-2 butanediol diether, PPG-10 1,6-hexanediol diether, PPG-12 1,6-hexanediol diether, PPG-14 hexanediol diether, PPG-20 hexanediol diether, and mixtures thereof. Preferred are those selected from the group consisting of PPG-10 1,4-butanediol diether, PPG-12 1,4-butanediol diether, PPG-10 1,6-hexandiol diether, and PPG-12 hexanediol diether, and mixtures thereof.

Examples of suitable alkoxylated diesters and trimesters are disclosed in U.S. Pat. Nos. 5,382,377, 5,455,025 and 5,597,555, assigned to Croda, Inc., and incorporated herein by reference.

Suitable lipids include C₈-C₂₀ alcohol monosorbitan esters, C₈-C₂₀ alcohol sorbitan diesters, C₈-C₂₀ alcohol sorbitan triesters, C₈-C₂₀ alcohol sucrose monoesters, C₈-C₂₀ alcohol sucrose diesters, C₈-C₂₀ alcohol sucrose triesters, and C₈-C₂₀ fatty alcohol esters of hydroxy acids. Examples of specific suitable lipids are sorbitan diisostearate, sorbitan dioleate, sorbitan distearate, sorbitan isosotearate, sorbitan laurate, sorbitan oleate, sorbitan palmitate, sorbitan sesquioleate, sorbitan esquistearte, sorbitan stearate, sorbitan triiostearte, sorbitan trioleate, orbitan tristeate, sucrose cocoate, sucrodilaurate, sucrose distearate, sucrose laurate, sucrose myristate, sucrose oleate, sucrose palmitate, sucrose ricinoleate, sucrose stearate, sucrose tribehenate, sucrose tristearate, myristyl lactate, stearyl lactate, isostearyl lactate, cetyl lactate, palmityl lactate, cocoyl lactate, and mixtures thereof.

Other emollients include mineral oil, petrolatum, cholesterol, dimethicone, dimethiconol, stearyl alcohol, cetyl alcohol, behenyl alcohol, diisopropyl adipate, isopropyl myristate, myristyl myristate, cetyl ricinoleate, sorbitan distearte, sorbitan dilaurate, sorbitan stearate, sorbitan laurate, sucrose laurate, sucrose dilaurate, sodium isostearyl lactylate, lauryl pidolate, sorbitan stearate, stearyl alcohol, cetyl alcohol, behenyl alcohol, PPG-14 butyl ether, PPG-15 stearyl ether, and mixtures thereof.

### Emulsifiers

The compositions of the invention may include emulsifiers in an amount of up to about 10%, preferably, in the amount of from about 0.5% to about 5%, by weight of the composition. Examples of emulsifiers include stearamidopropyl PG-dimonium chloride phosphate, stearamidopropyl ethyldimonium ethosulfate, stearamidopropyl dimethyl (myristyl acetate) ammonium chloride, stearamidopropyl dimethyl cetearyl ammonium tosylate, stearamidopropyl dimethyl ammonium chloride, stearamidopropyl dimethyl ammonium lactate, polyethylene glycols, polypropyleneglyocis, and mixtures thereof.

The compositions of the invention may include antidandruff agents, including zinc pyrithione, sulphur, and selenium sulfide. The compositions of the invention may include hair oxidizing/reducing agents such as hydrogen peroxide, perborate, thioglycolates and persulfate salts.

The compositions of the invention may include various thickeners, such as cross-linked acrylates, nonionic polyacrylamides, xanthan gum, guar gum, gellan gum, and the like; polyalkyl siloxanes, polyaryl siloxanes, and aminosilicones. Thickeners may be included in the amount of up to about 10%, preferably, in the amount of from about 0.2% to about 5% by weight of the composition.

The specific examples of the thickening silicon compounds include polydimethylsiloxane, phenylsilicone, polydiethylsiloxane, and polymethylphenylsiloxane. Suitable silicon compounds are described in European Patent Application EP 95,238 and U.S. Pat. No. 4,185,017, which are incorporated herein by reference. The compositions of the invention may also include silicone polymer materials such as described in U.S. Pat. No. 4,902,499, which is incorporated herein by reference.

The compositions of the invention may include hydrolyzed animal protein hair conditioning agents, such as Crotein Q-RTM (sold by Croda, Inc.). Other examples include urea, glycerol, and propoxylated glycerols, including those described in U.S. Pat. No. 4,976,953, which is incorporated by reference herein.

The compositions of the invention may include a hair setting agent to impart styling benefits upon application to hair, including homopolymers, copolymers, terpolymers, etc. For convenience in describing the polymers hereof, monomeric units present in the polymers may be referred to as the monomers from which they can be derived. The monomers can be ionic (e.g., anionic, cationic, amphoteric, zwitterionic) or nonionic. Examples of anionic monomers include unsaturated carboxylic acid monomers such as acrylic acid, methacrylic acid, maleic acid, maleic acid half ester, itaconic acid, fumeric acid, and crotonic acid; half esters of an unsaturated polybasic acid anhydride such as succinic anhydride, phthalic anhydride or the like with a hydroxyl group-containing acrylate and/or methacrylate such as hydroxyethyl acrylate and, hydroxyethyl methacrylate, hydroxypropyl acrylate and the like; monomers having a sulfonic acid group such as styrenesulfonic acid, sulfoethyl acrylate and methacrylate, and the like; and monomers having a phosphoric acid group such as acid phosphooxyethyl acrylate and methacrylate, 3-chloro-2-acid phosphooxypropyl acrylate and methacrylate, and the like.

The compositions of the invention may also include one or more absorbents, anti-acne agents, antiperspirants, anticaking agents, antifoaming agents, antimicrobial agents, antioxidants, antidandruff agents, astringents, binders, buffers, biological additives, buffering agents, bulking agents, chelating agents, chemical additives, coupling agents, conditioners, colorants, cosmetic astringents, cosmetic biocides, denaturants, drug astringents, detergents, dispersants, external analgesics, film formers, foaming agents, fragrance components, humectants, keratolytics, opacifying agents, pH adjusters, preservatives, propellants, proteins, retinoids, reducing agents, sequestrants, skin bleaching agents, skin-conditioning agents (humectants, miscellaneous, and occlusive), skin soothing agents, skin healing agents, softeners, solubilizing agents, lubricants, penetrants, plasticizers, salts, essential oils, and vitamins. The amount of each if used can vary widely depending on the product, however, the amount of each of these used will be less than about 50% by weight of the composition.

The examples of pH adjusters include sodium hydroxide, triethanoleamine, and aminomethylpropanol, and mixtures thereof. If pH adjusters are present in a personal care composition, the amount may vary from about 0.01% to about 5%. Most important is the fact that the pH which results is between about 4 and 10.

The examples of film formers include glycerin/diethylene glycol myrystate copolymer, glycerin/diethylene glycol adipate copolymer, ethyl ester of PVM/MA copolymer, PVP/dimethiconylacrylate/polycarbamyl/polyglycol ester, and mixtures thereof. If the film formers are present in the final product compositions, the amount may vary from about 0.1% to about 15.0% by weight of the composition.

The examples of vitamins include tocopherol, tocopherol acetate, retinoic acid, retinol, and retinoids.

The examples of anti-acne medicaments include resorcinol, sulfur, salicylic acid, erythromycin, zinc, and benzoyl peroxide.

The examples of skin bleaching or lightening agents include hydroquinone, and kojic acid. The examples of suitable aesthetic components such as fragrances, pigments, colorings, and the like, include panthenol and derivatives (e.g., ethyl panthenol), aloe vera, pantothenic acid and its derivatives, clove oil, menthol, camphor, eucalyptus oil, eugenol, menthyl lactate, witch hazel distillate, allantoin, bisabolol, and dipotassium glycyrrhizinate.

The composition of compounds of the present invention may be used as additives in other compositions.

### EXAMPLES

### Example 1

### Preparation of PPG-6 N-Methyldiethanolamine

Added to a clean, dry stirred tank pressure vessel with nitrogen inlet was 637.03 g (5.35 moles) of N-Methyldiethanolamine, a catalytic amount (5.55 g) of 45% KOH and 1.00 g of NaBH₄ as a color preservative. The vessel was then purged with nitrogen and heated to 110° C. Vacuum was applied for one hour to remove trace amounts of water, after which the temperature was raised to 130° C. Propylene oxide in an amount of about 1862.97 g (32.08 moles) was added at such a rate so the pressure remained below 50 psig. After all propylene oxide was added, the batch was left to react at constant pressure for an additional 4 hours after which the temperature was raised to 160° C. After two hours the temperature was lowered to 110° C. and full vacuum was applied for one hour. The reaction mixture was cooled to 50° C. and the catalyst was neutralized with 5.61 g of 50% Hypophosphorous Acid.

### Example 2

### Preparation of PPG-6 N-Methyldiethanolamine Di-Behenate

Added to a clean, dry stirred tank pressure vessel with nitrogen inlet and fitted with a distillation column was 288.71 g (0.62 moles) of the propoxylate from Example 1, 422 g (1.17 moles) of Behenic Acid (from Croda, Inc.) and a catalytic amount (1.75 g) of 50% Hypophosphorous Acid. The batch was heated to 220° C. under Nitrogen sparge and held for one hour, after which 100 mm Hg of vacuum was applied. During vacuum, the batch was monitored by measuring the acid value. The reaction was considered complete when the acid value was less than about 5 mg KOH.

### Example 3

### Methosulfate QUAT of PPG-6 N-Methyldiethanolamine Di-Behenate

Added to a clean, dry four neck round bottom flask fitted with Nitrogen inlet, mechanical stirrer, reflux condenser, temperature probe and drop funnel was 1275 g (1 mole) of the diester from Example 2. The batch was heated to 60-65° C. and 123 g (0.98 moles) of dimethyl sulfate was added drop-wise to maintain a temperature below 65° C. After about all the dimethyl sulfate was added, the batch was left to react for an additional hour after which the base value was checked. The reaction was considered complete when the base value was less than about 6 mg KOH. The final product was a waxy solid.

### Example 4

### Methosulfate QUAT of PPG-6 N-Methyldiethanolamine Di-Behenate and Incromine BD blend

Added to a clean, dry four neck round bottom flask fitted with Nitrogen inlet, mechanical stirrer, was 600 g of quat from Example 3 and 250 g of Incromine BD (available from Croda Inc.,). The batch was heated to 60-70° C. and stirred. The product was poured onto an aluminum sheet, allowed to cool, and flaked.

### Warehouse Storage Stability

Flaked samples of Example 3 (Flask A) and Example 4 (Flask B) were put in a stability oven set at 95° F. After 8 weeks, the material in Flask A had fused together while Flask B remained as individual flakes.

### Performance

Lotions with and without Example 3 Quat were prepared and evaluated for combing force reduction.

TABLE 1
Test Formulations

| Ingredients | Control | (i) |
|---|---|---|
| D.I. Water | 93.9 | 92.4 |
| Brij S-20-SO-(MH) | 1.2 | 1.2 |
| (Lot# 0000280756) | | |
| Crodacol 1618 | 4.8 | 4.8 |
| Neolone 950 | 0.1 | 0.1 |
| Preservative | | |
| Example 3 Quat | — | 1.5 |

TABLE 2
Wet Combing Evaluation Results (based on average of 10 tresses)

| | % Change of Combing | % Change of Total Work |
|---|---|---|
| Formulation | Force (gmf) | Done (Joules) |
| Control | +62.49 | +37.89 |
| Example 3 Quat | −84.62 | −88.76 |

The lotion with the quat from example 3 gave improved conditioning as evidenced by the significant decrease in combing force.

### Biodegradability and Aquatic Toxicity

Quat from example 3 was tested for biodegradability via method OECD 301B and Aquatic Toxicity via method OECD 202.

After 29 days the quat had biodegraded to 67%. The 48 hour EC₅₀ (aquatic toxicity) as measured on daphnia magna was >100 mg/l.

### Use

The products of Examples 1-4 can be used in a variety of personal care products such as those described herein.

### Conditioning Shampoo

| Ingredients | % |
|---|---|
| PART A | |
| Deionized Water | 32.42 |
| Glycerin | 1.00 |
| Disodium EDTA | 0.20 |
| PEG 7M | 0.20 |
| PART B | |
| Ammonium Lauryl Sulfate (ALS), 28% | 14.00 |
| Ammonium Lauryl Ether Sulfate (ALES), 25% | 40.00 |
| INCRONAM 30 (Cocamidopropyl Betaine) | 6.60 |
| Dimethicone | 1.22 |
| Glycol Distearate | 0.80 |
| Quat from Example 3 | 2.00 |
| PART C | |
| CRODASONE W (Hydrolyzed Wheat Protein PG-Propyl | 1.00 |
| Silanetriol) | |
| CROSILK LIQUID (Silk Amino Acids) | 0.20 |
| Methylisothiazolinone | 0.10 |
| PART D | |
| VERSATHIX (PEG-150 Pentaerythrityl Tetrastearate (and) | 0.26 |
| PPG-2 Hydroxyethyl Cocamide (and) Water) | |

### Procedure

The ingredients of Part A were combined and mixed until the solids dissolved. Part B ingredients were then added, and then mixed and heated to 75° C. until solids have dissolved. The A/B mix was allowed to cool to 40° C. and Part C ingredients were added. The pH was checked and adjusted, if necessary. Part D was then added with slow mixing. The pH was again checked and adjusted, if necessary, to between 5.5-6.5.

### Hair Conditioner

| Ingredients | % |
|---|---|
| PART A | |
| Deionized Water | 90.00 |
| Quat from Example 4 | 1.50 |
| Cetearyl Alcohol | 4.50 |
| PART B | |
| CRODAMOL SFX (PPG-3 Benzyl Ether 2-Etheyl Hexanoate) | 3.00 |
| Phenoxyethanol (and) Propylparaben (and) Butylparaben | 1.00 |
| (and) Methylparaben and Ethylparaben | |

### Procedure

Part A ingredients were combined heated to 80° C. with mixing. Mixing was continued and heating maintained for 10 minutes. The mixture was removed from heat and cooled to 40° C. Part B ingredients were added and mixed. The pH was adjusted to 5.5, if necessary, and mixed until smooth and homogeneous.

### Cationic Salt—Hair Conditioner

| Ingredients | % |
|---|---|
| PART A | |
| Deionized Water | 89.315 |
| Product from Example 2 | 1.50 |
| Incromine BD (Behenamidopropyldimethylamine) | 0.50 |
| Citric Acid | 0.185 |
| Cetearyl Alcohol | 4.50 |
| PART B | |
| CRODAMOL SFX (PPG-3 Benzyl Ether 2-Etheyl Hexanoate) | 3.00 |
| Phenoxyethanol (and) Propylparaben (and) Butylparaben | 1.00 |
| (and) Methylparaben and Ethylparaben | |

### Procedure

Part A ingredients were combined heated to 80° C. with mixing. Mixing was continued and heating maintained for 10 minutes. The mixture was removed from heat and cooled to 40° C. Part B ingredients were added and mixed. Adjust pH to between about 4-5, if necessary, and mixed until smooth and homogeneous.

Cationic Skin Lotion with Anti-Oxidant Botanical Extract

| Ingredients | % |
|---|---|
| PART A | |
| Deionized Water | 77.00 |
| CROMOLLIENT DP3A (Di-PPG-3 Myristyl Ether Adipate) | 5.00 |
| CRODAMOL SFX (PPG-3 Benzyl Ether 2 Ethyl Hexanoate) | 5.00 |
| Glycerin | 2.00 |
| CRODACOL S-70 (Stearyl Alcohol) | 4.00 |
| Quat from Example 4 | 3.50 |
| Citric Acid | 0.50 |
| PART B | |
| Propylene Glycol (and) Diazolidinyl Urea (and) Methylparaben | 1.0 |
| (and) Propylparaben | |
| PHYTOFLEUR FRENCH ROSE (Water (and) Butylene Glycol | 2.00 |
| (and) Rosa Gallica | |

### Procedure

Part A ingredients were mixed and heated to 75-80° C. The temperature was held for 15 minutes, and then cooled to 50° C. Part B ingredients were added individually with mixing. The pH was adjusted, if needed, to about 4.5-5.5 and cooled to the desired fill temperature.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A personal care product comprising at least one personal care ingredient and a diester having a biodegradability of at least about 50% as measured by a OECD-301B testing methodology, said diester having the structure of Formula (I), 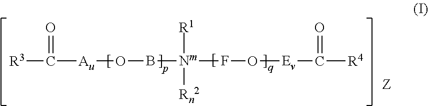
wherein R¹ and R² are independently selected from branched or straight chain, substituted or unsubstituted, saturated or unsaturated alkyl, cyclic or aromatic groups including between about 1 and about 22 carbon atoms;
B and F are independently a straight chain or branched alkyl group having between 2 and 8 carbon atoms,
p and q are independently 0 or 1;
A and E are independently selected from a straight chain or branched alkoxy group having between 1 and 8 carbon atoms;
u and v may be the same or different and are each an integer greater than 1;
R³ and R⁴ are independently selected from branched or straight chain, substituted or unsubstituted, saturated or unsaturated alkyl, cyclic or aromatic groups including between about 1 and about 36 carbon atoms;
Z is a counterion or salt former and included only in cases where the compounds of Formula (I) are a salt or quat as appropriate;
n is 0 or 1;
m is a lone pair of electrons or a positive charge;
with the proviso that if n is 0, then m is a lone pair of electrons and there is no counterion (Z); and if n is 1, then m is a positive charge and the counterion (Z) is present; and
with the proviso that if Aᵤ and Eᵥ each independently represent blocks containing propoxy and ethoxy groups, the number of propoxy groups is greater than the number of ethoxy groups.

2. The personal care product of claim 1, wherein R¹ and R² are independently selected from branched or straight chain, substituted or unsubstituted, saturated or unsaturated alkyl, cyclic or aromatic groups having between about 1 and about 10 carbon atoms.

3. The personal care product of claim 1, wherein R¹ and R² are independently selected from the group consisting of lower alkyl groups having between about 1 and about 8 carbon atoms.

4. The personal care product of claim 1, wherein R¹ and R² are independently selected from methyl, ethyl, n-propyl, iso-propyl, n-butyl, iso-butyl, or sec-butyl.

5. The personal care product of claim 1, wherein R³ and R⁴ are independently selected from saturated or unsaturated, straight chain or branched alkyl groups having between about 9 and 29 carbon atoms.

6. The personal care product of claim 1, wherein R³ and R⁴ are independently selected from saturated or unsaturated, straight chain or branched alkyl groups having between about 15 and 27 carbon atoms.

7. The personal care product of claim 1, wherein R³ and R⁴ are independently selected from unsaturated alkyl groups having between about 18 and about 24 carbon atoms, and having any degree of saturation.

8. The personal care product of claim 1, wherein B and F are independently a straight chain or branched alkyl group having between about 2 and about 4 carbon atoms.

9. The personal care product of claim 1, wherein A and E each independently represent blocks of alkoxy groups, where said blocks may be the same or mixed.

10. The personal care product of claim 1, wherein A and E each independently represent blocks of propoxy or ethoxy groups, wherein said propoxy groups may be linear or branched.

11. The personal care product of claim 10, wherein A and E represent blocks of ethoxy groups.

12. The personal care product of claim 10, wherein a ratio of propoxy groups to ethoxy groups ranges from about 3:2 to about 2:3.

13. The personal care product of claim 10, wherein a ratio of propoxy groups to ethoxy groups ranges from about 3:2 to about 4:1.

14. The personal care product of claim 1, wherein Aᵤ and Eᵥ are independently selected from the group having the Formula (III): 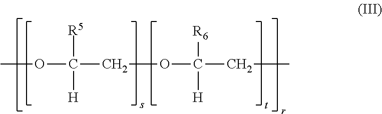
wherein R⁵ and R⁶ are independently H or CH₃;
s and t are independently 0 or an integer ranging from between 1 and 10; and
r is an integer greater than 1.

15. The personal care product of claim 14, wherein one of s or t is 0.

16. The personal care product of claim 14, wherein one of R⁵ or R⁶ is CH₃ and the other of R⁵ or R⁶ is H.

17. The personal care product of claim 14, wherein one of R⁵ or R⁶ is CH₃ and the other of R⁵ or R⁶ is H; one of s or t is 2 and the other of s or t is 3; and r is 1.

18. The personal care product of claim 14, wherein R⁵ is CH₃ and t is 0.

19. The personal care product of claim 1, wherein said diester has the structure of Formula (IV) 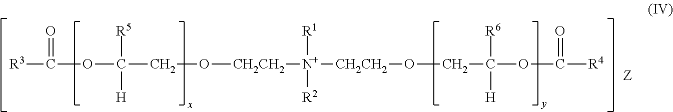
wherein R¹, R², R³, R⁴, and Z are as defined above;
R⁵ and R⁶ are independently H or CH₃;
x and y are independently an integer ranging from 1 to 10; and
Z is a counterion or salt-former.

20. The personal care product of claim 19, wherein x and y are independently an integer ranging from 2 to about 6.

21. The personal care product of claim 19, wherein x and y are independently an integer ranging from 2 to 5.

22. The personal care product of claim 19, wherein R⁵ and R⁶ are CH₃.

23. The personal care product of claim 19, wherein R³ and R⁴ are independently selected from saturated or unsaturated, straight chain or branched alkyl groups having between about 18 and 24 carbon atoms.

24. The personal care product of claim 19, wherein R⁵ and R⁶ are CH₃; R¹ and R² are independently methyl or ethyl; and R³ and R⁴ are independently selected from saturated or unsaturated alkyl groups having between 18 and 24 carbon atoms.

25. The personal care product of claim 19, wherein said diester contains at least one degree of unsaturation.

26. The personal care product of claim 1, wherein said diester has the structure of Formula (IVa) 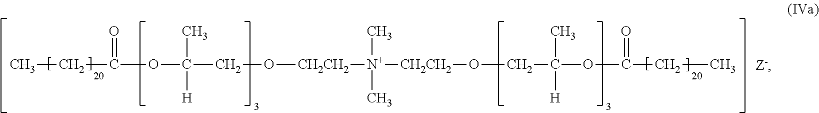
where Z is a counterion or salt-former.

27. The personal care product of claim 26, wherein said diester contains at least one degree of unsaturation.

28. The personal care product of claim 1, wherein said diester has the structure of Formula (IVb) 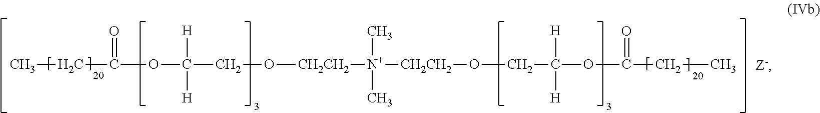
where Z is a counterion or salt-former.

29. The personal care product of claim 1, wherein R³ and R⁴ are selected from an unsubstituted alkyl group.

30. The personal care product of claim 1, wherein said diesters have an aquatic toxicity of at least about 10 mg/L as measured by a OECD-202: Daphnia sp. Acute Immobilization Test methodology.

31. The personal care product of claim 1, wherein an amount of said diester ranges from about 0.1% to about 20% on a cationic activity basis.

32. The personal care product of claim 1, wherein an amount of said diester ranges from about 0.5% to about 15% on a cationic activity basis.

33. The personal care product of claim 1, wherein said personal care ingredient is selected from the group consisting of cleaning agents, hair conditioning agents, skin conditioning agents, hair styling agents, antidandruff agents, hair growth promoters, perfumes, sunscreen compounds, pigments, moisturizers, film formers, humectants, alpha-hydroxy acids, hair colors, make-up agents, detergents, thickening agents, emulsifiers, antiseptic agents, deodorant actives and surfactants.

34. The personal care product of claim 1, wherein said personal care ingredient is a surfactant.

35. The personal care product of claim 1, wherein said product further comprises a solvent selected from the group consisting of water and lower alkanols and glycols having from 1 to 8 carbon atoms.

36. The personal care product of claim 1, wherein said product has a pH ranging from between about 4 to about 8.

37. The personal care product of claim 1, wherein said product has a pH ranging from between about 5 to about 7.

38. A diester having the structure of Formula (I), 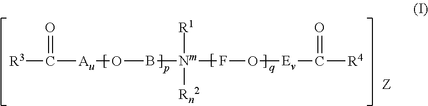
wherein R¹ and R² are independently selected from branched or straight chain, substituted or unsubstituted, saturated or unsaturated alkyl, cyclic or aromatic groups including between about 1 and about 22 carbon atoms;
B and F are independently a straight chain or branched alkyl group having between 2 and 8 carbon atoms,
p and q are independently 0 or 1;
A and E are independently selected from a straight chain or branched alkoxy group having between 1 and 8 carbon atoms;
u and v may be the same or different and are each an integer greater than 1;
R³ and R⁴ are independently selected from branched or straight chain, substituted or unsubstituted, saturated or unsaturated alkyl, cyclic or aromatic groups including between about 1 and about 36 carbon atoms;
Z is a counterion or salt former and included only in cases where the compounds of Formula (I) are a salt or quat as appropriate;
n is 0 or 1;
m is a lone pair of electrons or a positive charge;
with the proviso that if n is 0, then m is a lone pair of electrons and there is no counterion (Z); and if n is 1, then m is a positive charge and the counterion (Z) is present; and
with the proviso that if Aᵤ and Eᵥ each independently represent blocks containing propoxy and ethoxy groups, the number of propoxy groups is greater than the number of ethoxy groups.

39. The diester of claim 38, wherein R¹ and R² are independently selected from methyl, ethyl, n-propyl, iso-propyl, n-butyl, iso-butyl, or sec-butyl.

40. The diester of claim 38, wherein R³ and R⁴ are independently selected from unsaturated alkyl groups having between about 18 and about 24 carbon atoms, and having any degree of saturation.

41. The diester of claim 38, wherein A and E each independently represent blocks of alkoxy groups, where said blocks may be the same or mixed.

42. The diester of claim 38, wherein A and E each independently represent blocks of propoxy or ethoxy groups, wherein said propoxy groups may be linear or branched.

43. The diester of claim 38, wherein A and E represent blocks of ethoxy groups.

44. The diester of claim 38, wherein a ratio of propoxy groups to ethoxy groups ranges from about 3:2 to about 4:1.

45. The diester of claim 38, wherein Aᵤ and Eᵥ are independently selected from the group having the Formula (III): 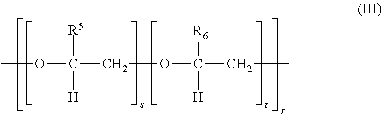
wherein R⁵ and R⁶ are independently H or CH₃;
s and t are independently 0 or an integer ranging from between 1 and 10; and
r is an integer greater than 1.

46. The diester of claim 45, wherein one of R⁵ or R⁶ is CH₃ and the other of R⁵ or R⁶ is H.

47. The diester of claim 45, wherein one of R⁵ or R⁶ is CH₃ and the other of R⁵ or R⁶ is H; one of s or t is 2 and the other of s or t is 3; and r is 1.

48. The diester of claim 38, wherein said diester has the structure of Formula (IV) 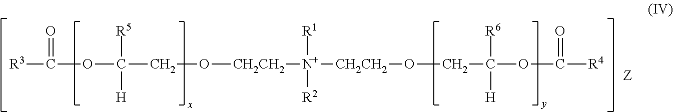
wherein R¹, R², R³, R⁴, and Z are as defined above;
R⁵ and R⁶ are independently H or CH₃;
x and y are independently an integer ranging from 1 to 10; and
Z is a counterion or salt-former.

49. The diester of claim 48, wherein x and y are independently an integer ranging from 2 to 5.

50. The diester of claim 48, wherein R⁵ and R⁶ are CH₃.

51. The diester of claim 48, wherein R⁵ and R⁶ are CH₃; R¹ and R² are independently methyl or ethyl; and R³ and R⁴ are independently selected from saturated or unsaturated alkyl groups having between 18 and 24 carbon atoms.

52. The diester of claim 38, wherein said diester contains at least one degree of unsaturation.

53. The diester of claim 38, wherein said diester has the structure of Formula (IVa) 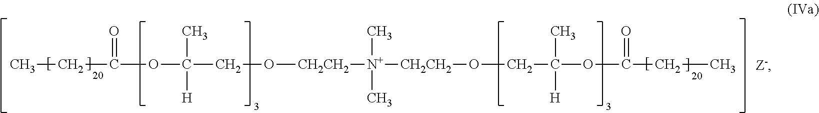
where Z is a counterion or salt-former.

54. The diester of claim 53, wherein said diester contains at least one degree of unsaturation.

55. The diester of claim 38, wherein said diester has the structure of Formula (IVb) 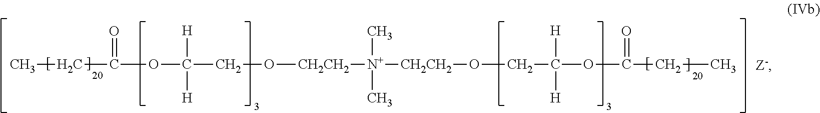
where Z is a counterion or salt-former.

56. A pastillatable composition comprising an alkylamidopropyl dimethylamine and a diester having the structure of Formula (I), 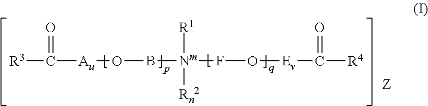
wherein R¹ and R² are independently selected from branched or straight chain, substituted or unsubstituted, saturated or unsaturated alkyl, cyclic or aromatic groups including between about 1 and about 22 carbon atoms;
B and F are independently a straight chain or branched alkyl group having between 2 and 8 carbon atoms,
p and q are independently 0 or 1;
A and E are independently selected from a straight chain or branched alkoxy group having between 1 and 8 carbon atoms;
u and v may be the same or different and are each an integer greater than 1;
R³ and R⁴ are independently selected from branched or straight chain, substituted or unsubstituted, saturated or unsaturated alkyl, cyclic or aromatic groups including between about 1 and about 36 carbon atoms;
Z is a counterion or salt former and included only in cases where the diesters of Formula (I) are a salt or quat as appropriate;
n is 0 or 1;
m is a lone pair of electrons or a positive charge;
with the proviso that if n is 0, then m is a lone pair of electrons and there is no counterion (Z); and if n is 1, then m is a positive charge and the counterion (Z) is present; and
with the proviso that if Aᵤ and Eᵥ each independently represent blocks containing propoxy and ethoxy groups, the number of propoxy groups is greater than the number of ethoxy groups.

57. A compound of Formula (V) 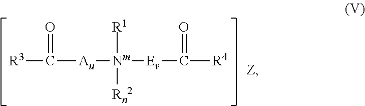
wherein R¹ and R² are independently selected from branched or straight chain, substituted or unsubstituted, saturated or unsaturated alkyl, cyclic or aromatic groups including between about 1 and about 22 carbon atoms;
A and E are independently selected from a straight chain or branched alkoxy group having between 1 and 8 carbon atoms;
u and v may be the same or different and are each an integer greater than 1;
R³ and R⁴ are independently selected from branched or straight chain, substituted or unsubstituted, saturated or unsaturated alkyl, cyclic or aromatic groups including between about 1 and about 36 carbon atoms;
Z is a counterion or salt former and included only in cases where the compounds of Formula (V) are a salt or quat as appropriate;
n is 0 or 1;
m is a lone pair of electrons or a positive charge;
with the proviso that if n is 0, then m is a lone pair of electrons and there is no counterion (Z); and if n is 1, then m is a positive charge and the counterion (Z) is present.

58. A personal care composition comprising: (1) a personal care ingredient;
(2) a diester having the structure of Formula (I), 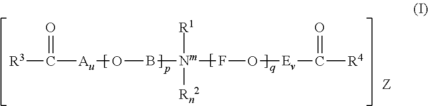
wherein R¹ and R² are independently selected from branched or straight chain, substituted or unsubstituted, saturated or unsaturated alkyl, cyclic or aromatic groups including between about 1 and about 22 carbon atoms;
B and F are independently a straight chain or branched alkyl group having between 2 and 8 carbon atoms,
p and q are independently 0 or 1;
A and E are independently selected from a straight chain or branched alkoxy group having between 1 and 8 carbon atoms;
u and v may be the same or different and are each an integer greater than 1;
R³ and R⁴ are independently selected from branched or straight chain, substituted or unsubstituted, saturated or unsaturated alkyl, cyclic or aromatic groups including between about 1 and about 36 carbon atoms;
Z is a counterion or salt former and included only in cases where the diesters of Formula (I) are a salt or quat as appropriate;
n is 0 or 1;
m is a lone pair of electrons or a positive charge;
with the proviso that if n is 0, then m is a lone pair of electrons and there is no counterion (Z); and if n is 1, then m is a positive charge and the counterion (Z) is present; and
with the proviso that if Aᵤ and Eᵥ each independently represent blocks containing propoxy and ethoxy groups, the number of propoxy groups is greater than the number of ethoxy groups; and
(3) a compound of Formula (V) 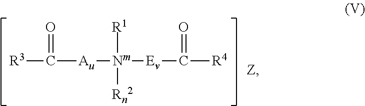
wherein R¹ and R² are independently selected from branched or straight chain, substituted or unsubstituted, saturated or unsaturated alkyl, cyclic or aromatic groups including between about 1 and about 22 carbon atoms;
A and E are independently selected from a straight chain or branched alkoxy group having between 1 and 8 carbon atoms;
u and v may be the same or different and are each an integer greater than 1;
R³ and R⁴ are independently selected from branched or straight chain, substituted or unsubstituted, saturated or unsaturated alkyl, cyclic or aromatic groups including between about 1 and about 36 carbon atoms;
Z is a counterion or salt former and included only in cases where the compounds of Formula (V) are a salt or quat as appropriate;
n is 0 or 1;
m is a lone pair of electrons or a positive charge;
with the proviso that if n is 0, then m is a lone pair of electrons and there is no counterion (Z); and if n is 1, then m is a positive charge and the counterion (Z) is present.

59. A compound PPG-6 Methydiethnolamine Di-Behenate and the quaternary ammonium salt thereof.

60. A personal care product comprising the compound of claim 59 and a personal care ingredient.

61. The product of claim 60, wherein said product is selected from the group consisting of a skin lotion, a hair conditioner, or a conditioning shampoo.
